(19) United States
(12) Patent Application Publication
(10) Pub. No.: US 2018/0184983 A1
(43) Pub. Date: Jul. 5, 2018

(54) METHOD FOR DETERMINING PHYSIOLOGICAL PARAMETERS FROM PHYSIOLOGICAL DATA

(71) Applicants: Lionsgate Technologies, Inc., Vancouver (CA); ESS Technology, Inc., Milpitas, CA (US)

(72) Inventors: Christian Leth PETERSEN, Burnaby (CA); A. Martin MALLINSON, Kelowna (CA)

(21) Appl. No.: 15/574,105

(22) PCT Filed: May 13, 2016
(86) PCT No.: PCT/CA2016/050553

§ 371 Date: Nov. 14, 2017

Related U.S. Application Data
(60) Provisional application No. 62/162,496, filed on May 15, 2015.

(51) Int. Cl. A61B 5/00; A61B 5/022; A61B 5/024; A61B 5/08

(52) U.S. Cl. CPC A61B 5/7278; A61B 5/0816; A61B 5/02416; A61B 5/022

(57) ABSTRACT

A method for determining a physiological parameter comprises receiving measured physiological data, parsing the measured physiological data into a plurality of time windows, each time window including a plurality of samples of the physiological data, fitting each of the plurality of time windows to a mathematical function utilizing a fitting function to obtain a plurality of sets of fit parameters, each set associated with a one of the plurality of time windows, and based on the plurality of sets of fit parameters, determining a physiological parameter.

### TECHNICAL FIELD

The present disclosure relates to determining physiological parameters from physiological data.

### BACKGROUND

Physiological parameters may be determined from physiological data that is obtained in a variety of different ways.

In an example, respiratory rate may be determined from a pulsatile photoplethysmographic (PPG) waveform measured utilizing, for example, pulse oximetry. The heart rate fluctuates during breathing, with an increase at inspiration and decrease at expiration, known as sinus arrhythmia. In addition, respiratory variations are also common in the pulsatile amplitude and the baseline (venous component) of the PPG signal. Thus, the PPG signal may be analyzed to extract any of heart rate fluctuation, pulsatile amplitude, and baseline, which may be utilized to determine the respiratory rate.

In another example, blood pressure may be determined from the oscillations in the measured pressure signal of an inflatable cuff that occludes blood flow through, for example, a patient's arm as the cuff pressure is increased and/or decreased. Systolic, diastolic and mean blood pressures can be estimated from the analysis of the shape of the oscillations in the pressure signal. Algorithms that perform such analysis are referred to as oscillometric algorithms.

Generally, noise and other artifacts present in the physiological data may reduce the accuracy of a determined a physiological parameter from the measured physiological data.

For example, the respiratory signals have been determined from wavelet analysis and morphology. In U.S. Pat. No. 8,880,576 to Ochs et al. morphology metric signals are utilized to extract information about respiration. In U.S. Pat. No. 7,035,679 to Addison et al. wavelet transforms are utilized to analyze the PPG waveform to remove artifacts and extract information such as the respiratory rate. However, these prior art methods are susceptible to noise and artifacts inherent in the PPG waveform, reducing the accuracy of the determined respiratory rate. Further, smearing in the time domain that may results from applying frequency domain methods like the wavelet transform may further degrade the accuracy of the prior art methods.

In another example, blood pressure may be determined by analyzing the pressure signal waveform in the time domain utilizing, for example, peak detection and peak based analysis to extract the pressure signal envelope. The quickly varying temporal content in a typical pressure signal makes analysis methods utilizing the frequency domain to determine blood pressure undesirable. The accuracy of time domain analysis based on the peaks in the pressure signal may be reduced by noise in the peak amplitudes caused by, for example, movement or other physical interferences, which may result in errant peak amplitudes and peak “troughs” with multiple offset readings. A number of prior art methods attempt to overcome this problem by means of peak fitting and peak-based filtering. Many peak based algorithms designed to suppress individual artifacts in the peaks, such as troughs and singular peak artifacts, have been reported in the literature. U.S. Pat. No. 5,704,362 to Hersh et al. discloses fitting a function curve to a plurality of oscillometric data values. However, even when fitting curves to the peak positions, the original noise in the peak amplitudes cannot be fully suppressed, introducing significant uncertainty in the blood pressure values determined from the peak positions.

The signal envelope extracted from the pressure signal may be analyzed utilizing, for example, an oscillometric algorithm to determine the systolic, diastolic and mean pressure readings. However, the use of oscillometric algorithms is complicated by the poor resolution of the envelope determined by the prior art methods and, as a result, many prior art implementations of oscillometric algorithms utilize primitive threshold-based methods as described in, for example, Sapinski (Med. & Biol. Eng. & Comput. 30 671 1992).

Improvements to determining physiological parameters based on physiological data are desired.

### DRAWINGS

The following figures set forth embodiments in which like reference numerals denote like parts. Embodiments are illustrated by way of example and not by way of limitation in the accompanying figures.

FIG. 1 is a block diagram of a system for determining a physiological parameter from raw physiological data according to an embodiment;

FIG. 2 is a flow chart of a method of determining a physiological parameter from raw physiological data according to another embodiment shown in FIG. 1; and

FIG. 3 is a graph of raw photoplethysmographic (PPG) data and a sinusoidal function fit to the PPG data according to another embodiment;

FIG. 4 is a graph of the offset parameter and the delta of the sinusoidal function shown in FIG. 3;

FIG. 5 a block diagram of a blood pressure extraction system according to another embodiment;

FIG. 6 is a block diagram of a signal quality logic element utilized in the blood pressure extraction system shown in FIG. 5;

FIG. 7 is a graph of blood pressure data and a sinusoidal function generated by a fit to the blood pressure data according to another embodiment; and

FIG. 8 is a graph of the time variation of the amplitude parameter of the sinusoidal function shown in FIG. 7.

### DETAILED DESCRIPTION

The following describes a method for determining physiological parameters from oscillatory physiological data. For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

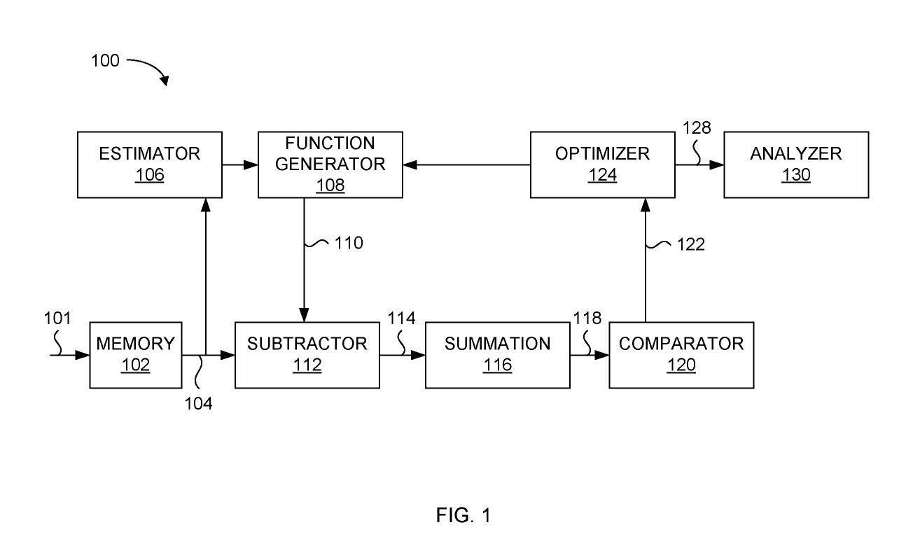

Referring to FIG. 1, a system 100 for determining a physiological parameter from raw physiological data 101 is shown.

The raw physiological data 101 is saved in a memory 102 as buffered data 104. The buffered data 104 stored in the memory 102 is parsed into time windows, where the length of the time window of data is predetermined to include a sufficiently large number of samples to fit each time window of data. In an embodiment, the time windows overlap. For example, the time windows may be a sliding time window that is updated on a sample by sample basis such that a time window comprising n samples will have an overlap of n−1 samples with a previous time window.

In some embodiments, an absolute time may be associated with the fitted parameters from each time window. For example, when measuring blood pressure, an absolute time may be used to correlate the time of an event in the fitted parameters to the pressure in the cuff at the time of that event. Correlation to the absolute time of the event as ultimately recorded by the parameters may assured by providing a constant delay from the input to the output of the system. The absolute time associated with a window may be the start time, midpoint or end time of the window.

A time window of buffered data 104 is a sequence of samples of the raw physiological data 101, y₁, y₂, . . . , yₙ. Each time window of the buffered data 104 is fit to a mathematical function, ƒ(tₙ), utilizing an iterative process using a many-parameter least squares fit. In some embodiments, each window may be selected to cover at least one period of the mathematical function. In other embodiments, each time window may be selected to cover less than a period of the mathematical function.

The values 110 of the mathematical function at times tₙ are generated by the function generator 108 based on the inputted parameters of the mathematical function. During a first iteration of the fit of an initial time window of buffered data 104, the buffered data 104 being fit may be provided to an optional estimator 106 that estimates the initial value of one or more parameters of the mathematical function. For example, in an embodiment in which the mathematical function is a sinusoidal function, the estimator 106 may estimate a frequency parameter using either time domain methods such as peak detection or frequency domain methods such as the Fast Fourier Transform (FFT), or any other frequency estimation technique. If an estimator is not utilized, the initial parameters of the mathematical function may be determined by a population based average of the physiological data.

The values 110, as well as the time window of buffered data 104 are input into subtractor 112, which determines, for each sample, yₙ, of the buffered data 104, a difference between the sample value and the value of the mathematical function, ƒ(tₙ). The difference values 114 are sent to summation element 116 that determines a sum 118 based on the sums the squares of the differences between each sample and the associated value 110:

Σₙ₌₁^{N}[yₙ−ƒ(tₙ)]² (1)

The sum 118 is sent to a comparator 120 which compares the sum 118 to a predetermined condition to determine whether the function parameters result in a sufficient fit between the mathematical function and the time window buffered data 104. The condition may be, for example, that the sum 118 is less than a threshold value.

If the comparator 120 determines that the sum 118 does not meet the condition, then a signal 122 sent to the optimizer 124 instructing the optimizer 124 to modify the function parameters and send the modified function parameters 126 to the function generator 108 for a subsequent iteration of fitting. The iterations are repeated until the comparator 120 determines that the sum 118 meets the condition.

If the comparator 120 determines that the sum 118 meets the condition, then the signal 122 instructs the optimizer to output the function parameters last input into the function generator 108 as fit parameters 128. The fit parameters 128 are sent to an analyzer 130 which determines one or more physiological parameters utilizing the fit parameters 128. The analyzer 130 may include a memory (not shown) to store the fit parameters 128 from fittings of a plurality of time windows of buffered data 104 in order to determine physiological parameters based on the time variation of the fit parameters 128.

An optional counter (not shown) may determine the number of iterations performed for the sum 118 to meet the condition for a given time window of buffered data 104. The number of iterations may be compared with the number of iterations for the fit of a previous time window to determine a sudden increase in the number of iterations performed before the sum 118 meets the condition. A sudden increase in the iterations is an indication that the waveform has changed shape that can be used as a signal quality indicator (SQI). The determination of number of iterations performed may indicate an additional signal is present in the data. For example, a specific periodicity in the number of iterations required to meet the condition for a given time window may indicate regular breathing, movement or other significant physiological aspect.

In some embodiments, the fit parameters 128 may also be sent to the function generator 108 for use as initial parameters for the mathematical function during fitting of the next time window of buffered data 104. Utilizing previously determined fit parameters 128 as initial parameters for the next fit may reduce the number of iterations performed before the sum 118 is determined to meet the condition, reducing the overall time and processing resources utilized for the fit.

In some embodiments, the mathematical function utilized by the function generator 108 is a sinusoidal function. For example, the sinusoidal function may have the form:

ƒ(tₙ)=A cos(ωtₙ+θ)+C (2)

where A is the amplitude parameter, ω is the angular frequency parameter, θ is the phase shift parameter, and C is the offset parameter. Each time window that is fit to the mathematical function has an associated set of fit parameters.

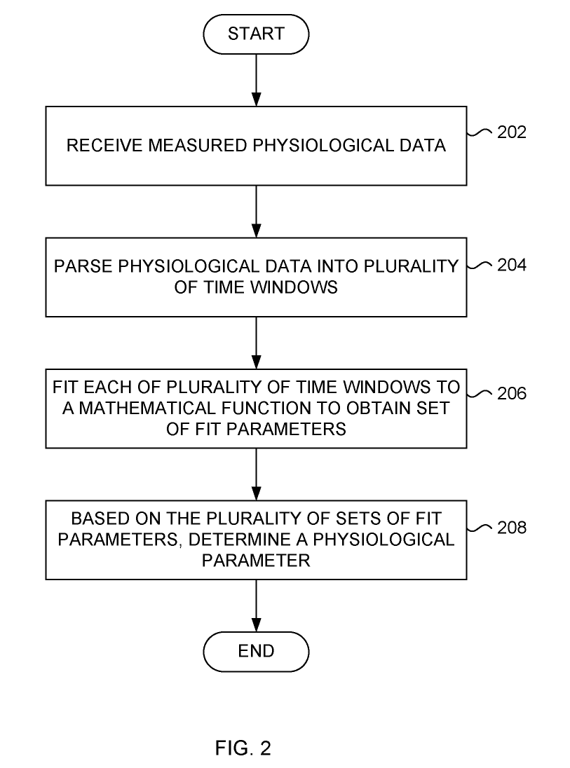

Referring to FIG. 2, a flow chart illustrating a method of determining a physiological parameter from raw physiological data is shown. The method shown in FIG. 2 may be performed by, for example, the system 100 shown in FIG. 1. At 202, raw physiological data is received. Receiving the data may include storing the data in a buffer or memory, such as memory 102. At 204, the raw physiological data is parsed into a plurality of time windows. At 206, each time window of physiological data is fit to a mathematical function utilizing a many-parameter least squares fit in order to determine a set of fitted parameters for each time window. At 208, the plurality of sets of fitted parameters associated with the plurality of time windows are analyzed to determine one or more physiological parameters. Analyzing at 208 may include determining a time variation of one or more of the fitted parameters.

In a first embodiment, the physiological data is pulsatile photoplethysmographic (PPG) data measured by, for example, a pulse oximeter. Because PPG data is oscillatory, the PPG data may be fit utilizing the sinusoidal mathematical function of equation 2 described above. The size of the time windows in this embodiment may be selected to be in the range of 1-2 heart beats, or about 1-2 seconds for typical resting heart rates.

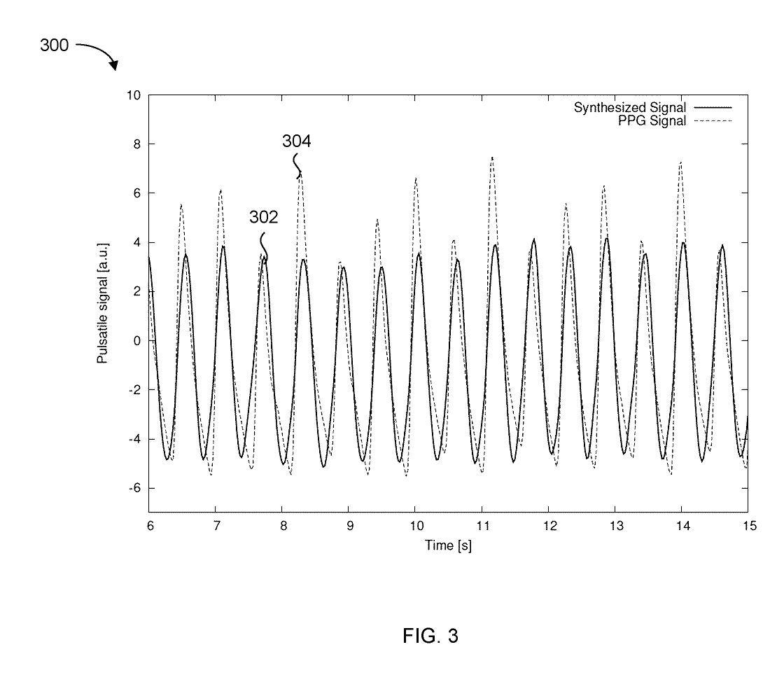

Referring to FIG. 3, a graph 300 of an example of a fitted waveform 302 generated by fitting a sinusoidal function to the oscillatory raw PPG data 304 is shown. The noise in the peak amplitudes and the non-uniform shape of the pulsatile structures in the raw PPG data 304 are not present in the fitted waveform 302.

The amplitude parameter A, angular frequency parameter w, and the offset parameter C of the fit parameters associated with the fitted waveform 302 have time variations that are associated with a respiration rate. In addition, the difference (delta) between the raw PPG data 304 and the fitted waveform 302 may also exhibit time variations that are associated with respiration.

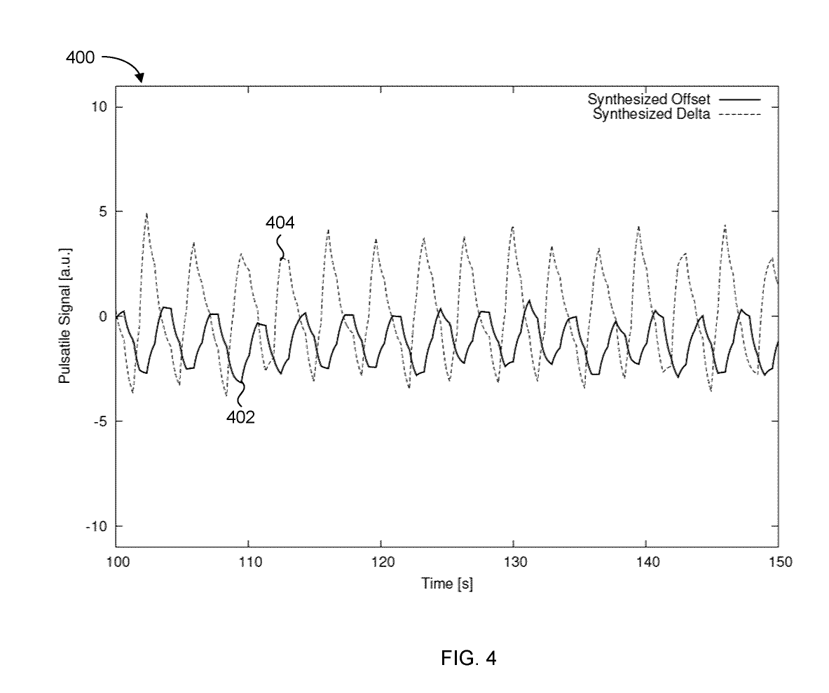

FIG. 4 shows a graph 400 of the time variation of the offset parameter C 402 and the delta 404 of the fitted waveform 302 of FIG. 3. The time variation of the offset parameter C 402 and the delta 404 of the fitted waveform 302 show a period that is comparable to the respiratory period. The signals have phase and amplitude difference, which are dependent on the physical coupling between each parameter and the respiratory effort of the patient.

In an embodiment, a phased array feedback system may be utilized to extract the respiratory rate from the fitted parameters. The phased array feedback system may be a component of, for example, the analyzer 130 shown in FIG. 1. The phased array feedback system aggregates respiratory components (or other physiological parameters of interest) from multiple noisy physiological data signals, such as multiple PPG signals. The respiratory component of each PPG signal may have a amplitude and phase that differs from the amplitude and phase of the respiratory components of the other PPG signals. The phased array feedback system adjusts the phase and amplitude of each respiratory component to facilitate constructively adding the respiratory components into a single aggregate respiration signal. The respiratory rate may be determined by the oscillation in the aggregate respiration signal, and may be extracted by means of time-domain analysis (e.g. peak detection) or frequency domain analysis (e.g. Fourier transform).

In a second embodiment, the physiological data is pressure data measured by, for example, an inflatable cuff.

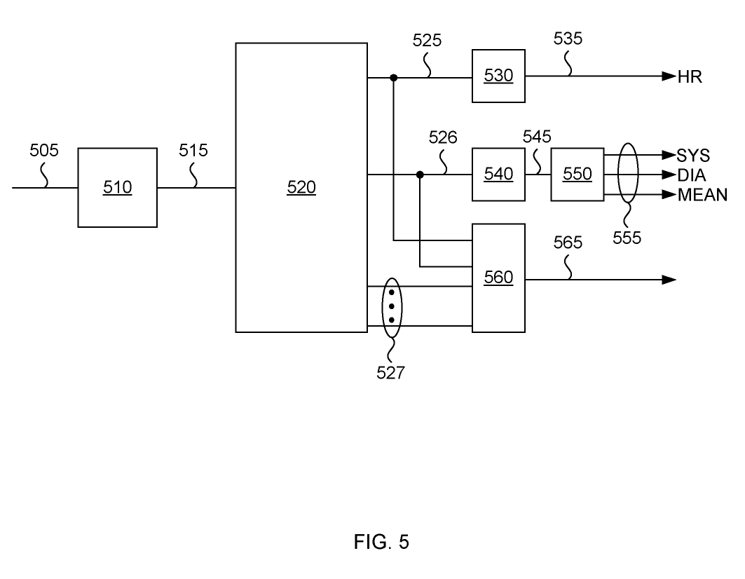

Referring to FIG. 5, a block diagram of an example blood pressure extraction system 500 is shown. The blood pressure extraction system 500 receives raw pressure data 505 from, for example, a pressure cuff (not shown). The raw pressure data 505 may be passed through a filter 510 to remove a DC component from the raw data 505 to generate filtered pressure data 515. The filter 510 may be a high pass filter utilizing filtering techniques such as, for example, time domain filtering including moving averages, exponential moving averages, and FIR filtering, or frequency domain filtering such as fast Fourier transforms, or a combination of time domain and frequency domain filtering techniques.

The filtered pressure data 515 is input to a function fitting element 520 which performs the window based fitting of the filtered blood pressure data 515 to a mathematical function to determine fitted parameters. Because pressure data measured by an inflatable cuff is oscillatory, the function fitting element 520 may fit the filtered blood pressure data 515 utilizing the sinusoidal mathematical function of equation 2 described above. The function fitting element 520 may perform the functions of the memory 102, the estimator 106, the function generator 108, the subtractor 112, the summation element 116, the comparator 120, and the optimizer 124 described above with regard to the example system 100 shown in FIG. 1. The size of the time windows utilized by the function fitting element 520 may be selected to be in the range of 1-2 heart beats, or about 1-2 seconds.

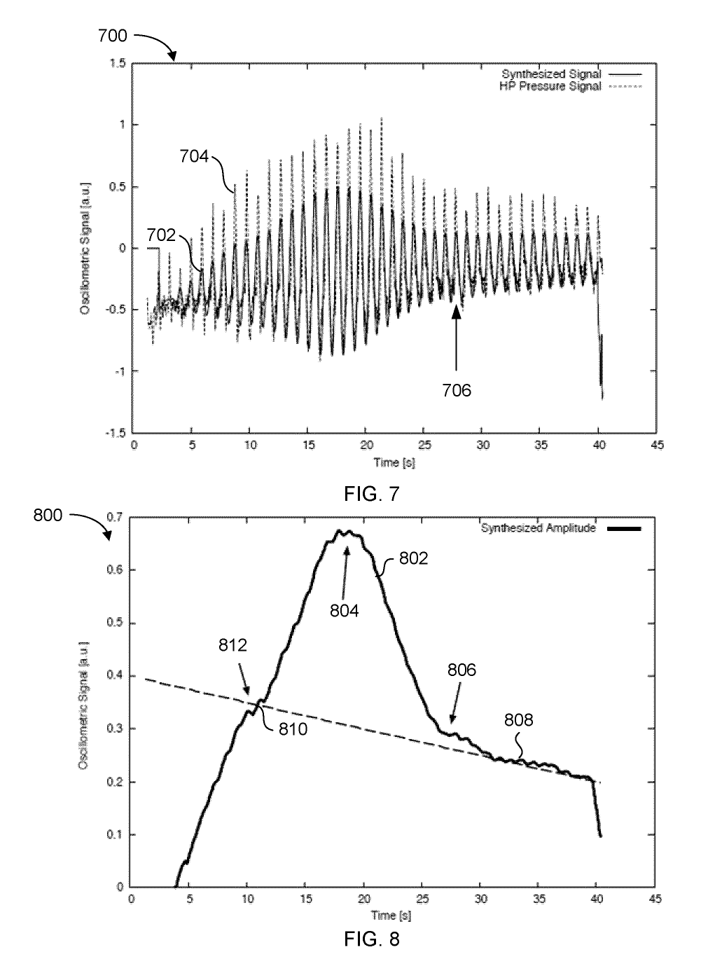

The fitted parameters associated with each fitted time window are output by the function fitting element 520 for further analysis. For example, the fitted angular frequency parameters, ω, are output as frequency signal 525, which is input to a frequency detection element 530 to determine the heartrate 535. The fitted amplitude parameters, A, are output as amplitude signal 526, which may be passed through a filtering element 540, such as for example a low-pass filter, and a filtered amplitude signal 545 is input into a blood pressure extraction element 550. The blood pressure output 555 from the blood pressure extraction element 550 may include the systolic pressure SYS, the diastolic pressure, DIA, and the mean pressure MEAN. The determination of the blood pressure output 555 from the filtered amplitude signal 245 is described in more detail below with reference to FIGS. 7 and 8.

The function fitting element 520 may also generate other outputs 227, which may include, for example, the fitted phase parameters, θ, the fitted offset parameters, C, as well as other values such as the number of iterations for each fit, and the root-mean square (RMS) error of the fit. The frequency signal 525, the amplitude signal 225, as well as the other outputs 527 of the function fitting element 520, are input to a signal quality logic element 560. The signal quality logic element 560 compares one or more of the inputs 525, 526, and 527 to a condition to determine whether an error has occurred, in which case an error output 565 is generated. The ERROR signal 565 may indicate, for example, whether the raw pressure data 505 input into the blood pressure extraction system 500 is determined to be suitable for determining physiological parameters.

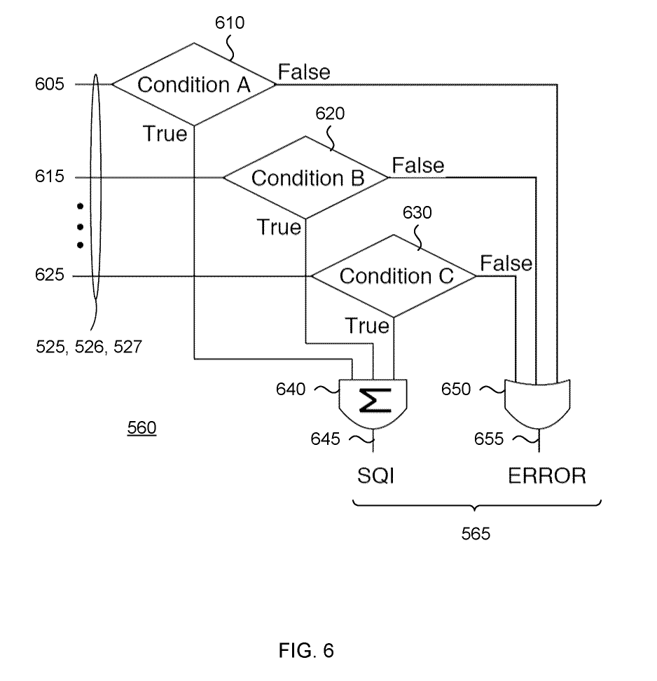

Referring to FIG. 6, a functional diagram of one embodiment of a signal quality logic element 560 is shown. In the example signal quality logic element 560 shown, three input signals from the outputs 525, 256, and 527 of the function fitting element 520 are utilized. A first signal 605 is compared to a first condition, condition A, by a first element 610, a second signal 615 is compared to a second condition, condition B, by a second element 620, and a third input 625 is compared to third condition, condition C, by a third element 630. The elements 610, 620, 630 each output a signal at a FALSE output if the signal does not meet the respective condition, and each output a signal at a TRUE output if the signal meets the respective condition.

In the example shown, the FALSE outputs may be provided to an OR logic element 650 which generates an ERROR flag 655 which indicates that one or more of the signals 605, 615, 625 do not meet the condition. The TRUE outputs are input to a summation element 640, which provides a signal quality indicator (SQI) output 645. The SQI output 645 may be utilized indicate a confidence in the raw pressure data 505 input into the blood pressure extraction system 500, with a higher SQI output 645 indicate greater confidence.

Examples of signals and conditions that may be utilized by the signal quality logic element 560 include: the fitted frequency parameter being in a physiologically possible range for a heart rate, for example between 0.5 and 4 Hz; the fitted amplitude parameter meeting or exceeding a threshold amplitude; a number of iterations to reach convergence exceeding a threshold number; and a sudden change of any of the signal values such as, for example, a sudden increase in the RMS error output.

Referring to FIG. 7, graph 700 shows an example of the waveform 702 generated by the fitting parameters determined by fitting the sinusoidal function of equation 2 to raw blood pressured data 704 sampled at 40 Hz. Graph 700 shows that the noise present in the peak amplitudes of the raw data 704 is reduced in the waveform 702. For example, the peaks of the raw pressure 704 in the vicinity of maximum of envelope located in the time range from 15 s to 20 s are spurious, whereas the spuriousness in the same time range is suppressed in the waveform 702. Further, the reduced noise in the waveform 702 compared to the raw pressure data 704 facilitates identifying a bend in the amplitude, identified as a constriction in the envelope of the waveform 702 at approximately time=28 s and identified by arrow 706. The bend may be utilized to determine the diastolic pressure (e.g. in conjunction with timing information of cuff pressure).

The fitted amplitude parameter defines the envelope of the waveform 702. Referring to FIG. 8, a graph 800 of the fitted amplitude parameters 802 of the exemplary waveform 702 shown in FIG. 7 is shown. The fitted amplitude parameters 802 include a peak 804, which may be utilized to determine the mean blood pressure.

The diastolic pressure may be identified by the sudden change, as indicated by arrow 806, in the first derivative of the fitted amplitude parameter 802, which corresponds with the bend discussed with reference to FIG. 7. In an example, the diastolic pressure is determined when a second derivative of the fitted amplitude 802 meets or exceeds a threshold. A baseline 808 may be determined utilizing a portion of the fitted amplitude parameter 802 that trails the sudden change 806. A linear method may be utilized to determine the systolic pressure by, for example, linearly extrapolating the baseline 808 back to an intersection 810 with the fitted amplitude parameter 802. The intersection 810 may be utilized to determine the systolic pressure. The location of the systolic pressure is also marked by a disturbance 812 in the fitted amplitude parameter 802, which is of smaller magnitude than the sudden change 806. In some embodiments, the disturbance 812 may be utilized to determine the systolic pressure, or may be utilized to verify the determination of the systolic pressure utilizing linear extrapolation of the baseline 808. The disturbance 812 may be determined when a second derivative of the fitted amplitude parameter 802 meets or exceeds a second threshold.

In an alternative embodiment, rather than analyzing the fitted amplitude parameters, the fitted parameters may be utilized in an oscillometric algorithm rather than the raw data. Because of the reduction in the noise of the fitted waveform compared to the raw data, utilizing the fitted parameters in an oscillometric algorithm will result in better blood pressure estimates compared to utilizing the raw data.

Disclosed is a method for determining a physiological parameter from measured physiological data in which the physiological parameter is determined based on the fitted parameters generated through fitting the physiological data to a mathematical function utilizing a least squared fit. By utilizing the fitted parameters rather than the physiological data, the effect of noise and other artifacts that may be present in the measured physiological data is reduced resulting in a better determination of the physiological parameter.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A method for determining a physiological parameter, the method comprising:
receiving measured physiological data;
parsing the measured physiological data into a plurality of time windows, each time window including a plurality of samples of the physiological data;
fitting each of the plurality of time windows to a mathematical function utilizing a fitting function to obtain a plurality of sets of fit parameters, each set associated with a one of the plurality of time windows; and
based on the plurality of sets of fit parameters, determining a physiological parameter.

2. The method according to claim 1, further comprising determining the time variation of the fit parameters from the plurality of sets of fit parameters, and wherein determining the physiological parameter is based on the time variation of the fit parameters.

3. The method according to claim 1, wherein a first set of fit parameters obtained from fitting a first time window fit are used as an initial set of fit parameters for fitting a subsequent second time window.

4. The method according to claim 1, wherein a size of each of the time windows is at least one period of the mathematical function.

5. The method according to claim 1, wherein the physiological data is photoplethysmographic (PPG) data, and the physiological parameter determined is at least a respiratory rate.

6. The method according to claim 5, wherein a size of each time window is predetermined to be in the range of one of 1-2 heartbeats or 1-2 seconds.

7. The method according to claim 5, wherein the mathematical function is a generalized sinusoidal waveform of the form:
ƒ(tₙ)=A cos(ωtₙ+θ)+C
where A is the amplitude parameter, ω is the angular frequency parameter, θ is the phase shift parameter, and C is the offset parameter.

8. The method according to claim 7, further comprising:
determining an estimated initial frequency of the PPG data utilizing a frequency-estimation algorithm; and
utilizing the estimated initial frequency as the frequency parameter for the first iteration of the many-parameter least squares fit.

9. The method according to claim 7, further comprising:
determining an estimated initial phase of the PPG data utilizing a phase-estimating algorithm; and
utilizing the estimated initial phase as the phase parameter input for a first iteration of a many-parameter least squares fit.

10. The method according to claim 7, further comprising determining a time signal quality index of the fitting of each time window, the time signal quality index determined by at least one of:
the number of iterations required for the sum of the squared differences to be less than a sum threshold;
the amplitude parameter meeting or exceeding a amplitude threshold;
the root mean squared (RMS) value of the fitting function; and
the change in any fit parameter compared with the fit parameters associated with a previous time window meeting or exceeding a change threshold.

11. The method according to claim 10, wherein the physiological data is pressure data measured by an oscillometric cuff, and the physiological parameter determined is at least one of a systolic pressure, a diastolic pressure, a mean pressure, and a heart rate.

12. The method according to claim 10, wherein a size of each time window is predetermined to be in the range of one of 1-2 heartbeats or 1-2 seconds.

13. The method according to claim 10, wherein the mathematical function is a generalized sinusoidal waveform of the form:
ƒ(tₙ)=A cos(ωtₙ+θ)+C
where A is the amplitude parameter, ω is the angular frequency parameter, θ is the phase shift parameter, and C is the offset parameter.

14. The method according to claim 13, wherein the systolic pressure and the diastolic pressure are determined based on a time variation of the amplitude parameters of the plurality of sets of fitting parameters.

15. The method according to claim 13, wherein the heart rate is determined by the frequency parameter of the plurality of sets of fitted parameters.

16. The method as claimed in claim 10, wherein a size of each time window is in a range of either 1-2 heartbeats or 1-2 seconds.

17. The method according to claim 13, further comprising determining a time signal quality index of the fitting of each time window, the time signal quality index determined by at least one of:
the number of iterations required for the sum of the squared differences to be less than a sum threshold;
the amplitude parameter meeting or exceeding a amplitude threshold;
the root mean squared (RMS) value of the fitting function; and
the change in any fit parameter compared with the fit parameters associated with a previous time window meeting or exceeding a change threshold.

18. The method of claim 13, further comprising:
low pass filtering the amplitude parameters of the plurality of sets of fit parameters, the low pass filtering having a kernel size of about one heartbeat; and
determining the mean blood pressure as the maximum of the low pass filtered amplitude parameters.

19. The method as set forth in claim 13, in which determining the diastolic pressure comprises:
low pass filtering the amplitude parameters of the plurality of sets of fit parameters, the low pass filtering having a kernel size of about one heartbeat;
determining a diastolic pressure a second derivative of the low pass filtered amplitude parameter meeting a threshold.

20. The method of claim 19, further comprising:
determining a baseline of a portion of the low pass filtered amplitude parameters at times after the time of the determined diastolic pressure point;
determining an intersection of the baseline to the low pass filtered amplitude parameters by extrapolating the baseline; and
identifying the intersection as the systolic pressure.

21. The method as set forth in claim 10, wherein determining at least one of a systolic pressure, a diastolic pressure, a mean pressure, and a heart rate comprises inputting the plurality of sets of fit parameters into a peak-based oscillometric algorithm.
